(19) United States
(12) Patent Application Publication
(10) Pub. No.: US 2010/0036173 A1
(43) Pub. Date: Feb. 11, 2010

(54) PRODUCTION OF BUTENES AND DERIVATIVES THEREFROM FROM AQUEOUS ETHANOL

(75) Inventors: Leo Ernest Manzer, Wilmington, DE (US); Michael B. D'amore, Wilmington, DE (US); Edwark S. MIller, JR., Knoxville, TN (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. DU PONT DE NEMOURS AND COMPANY, Wilmington, DE (US)

(21) Appl. No.: 12/513,700

(22) PCT Filed: Nov. 30, 2007
(86) PCT No.: PCT/US2007/024669

§ 371 Date: Oct. 19, 2009

Related U.S. Application Data
(60) Provisional application No. 60/879,364, filed on Jan. 9, 2007.

(51) Int. Cl. C07C 41/05; C07C 29/04; C07C 1/20

(52) U.S. Cl. 568/697; 568/895; 585/322; 585/323; 585/324; 585/331

(57) ABSTRACT

The present invention relates to the production of butenes and derivatives thereof from aqueous ethanol, optionally obtained from a fermentation broth. The butenes thus produced find use as intermediates for the production of polyethylenes and for the production of other known, useful materials.

### FIELD OF THE INVENTION

The present invention relates to the production of butenes and derivatives thereof from aqueous ethanol, optionally obtained from a fermentation broth.

### BACKGROUND

Efforts directed at improving air quality and increasing energy production from renewable resources have resulted in renewed interest in alternative fuels, such as ethanol and butanol, that might replace gasoline and diesel fuel, or be additives in these fuels as well as others.

It is known that ethanol can be recovered from a number of sources, including synthetic and fermentation feedstocks. Synthetically, ethanol can be obtained by direct catalytic hydration of ethylene, indirect hydration of ethylene, conversion of synthesis gas, homologation of methanol, carbonylation of methanol and methyl acetate, and synthesis by both homogeneous and heterogeneous catalysis. Fermentation feedstocks can be fermentable carbohydrates (e.g., sugar cane, sugar beets, and fruit crops) and starch materials (e.g., grains including corn, cassava, and sorghum). When fermentation is used, yeasts from the species including Saccharomyces can be employed, as can bacteria from the species Zymomonas, particularly Zymomonas mobilis. Ethanol is generally recovered as an azeotrope with water, so that it is present at 95.57 weight percent with respect to the weight of water and ethanol combined. See Kosaric, et. al, Ullmann's Encyclopedia of Industrial Chemistry, Sixth Edition, Volume 12, pages 398-473, Wiley-VCH Verlag GmbH & Co. KGaA, Weinheim, Germany, and P. L. Rogers, et al., Adv. Biochem. Eng. 23 (1982) 27-84.

Methods for producing 1-butanol are known. It is known that 1-butanol can be prepared by condensation from ethanol over basic catalysts at high temperature using the so-called “Guerbet Reaction.” See for example, J. Logsdon in Kirk-Othmer Encyclopedia of Chemical Technology, John Wiley and Sons, Inc., New York, 2001.

Some references further describing the production of 1-butanol from ethanol include: Chinese Pat. No. CN 12168383C; C. Yang and Z. Meng, J. of Catalysis (1993), 142 (1), 37-44; A. S. Ndou, N. Plint, and N. J. Coville, Applied Catalysis, A: General (2003), 251 (2), 337-345; T. Takahashi, Kogyo Kagaku Zasshi (1946), 49 113-114; T. Takahashi, Kogyo Kagaku Zasshi (1946), 49 114-115; V. Nagarajan, N. R. Kuloor, Indian Journal of Technology (1966), 4 (2), 46-54; V. Nagarajan, Chemical Processing & Engineering (Bombay) (1970), 4 (11), 29-31, 38; V. Nagarajan, Indian Journal of Technology (1971), 9 (10), 380-386; V. Nagarajan, Chemical Processing & Engineering (Bombay) (1971), 5 (10), 23-27; K. W. Yang, X. Z. Jiang and W. C. Zhang, Chinese Chemical Letters (2004), 15 (112), 1497-1500; K. Yang, W. Zhang, and X. Jiang, Chinese Patent No. 1528727 (assigned to Zhejiang Univ.); C. A. Radlowski and G. P. Hagen, U.S. Pat. No. 5,095,156 (assigned to Amoco Corp.); C. Y. Tsu and K. L. Yang, Huaxue (1958), (No. 1), 39-47; B. N. Dolgov and Yu. N. Volnov, Zhurnal Obshchei Khimii (1993), 3 313-318; M. J. L. Gines and E. Iglesia, J. of Catalysis (1998), 176 (1), 155-172; T. Tsuchida, A K. Atsumi, S. Sakuma, and T. Inui, U.S. Pat. No. 6,323,383 (assigned to Kabushiki Kaisha Sangi); and GB Pat. No. 381,185, assigned to British Industrial Solvents, Ltd.

Butenes are useful intermediates for the production of linear low density polyethylene (LLDPE) and high density polyethylene (HDPE), as well as for the production of transportation fuels and fuel additives. The bulk of butenes (1-butene, 2-butene, isobutene) are currently produced as byproducts in the refining of motor fuel, and from the various cracking processes of butane, naphtha, or gas oil (Weissermel, K. and Arpe, H.-J. (translated by Lindley, C. R. and Hawkins, S.) in Industrial Organic Chemistry, 4^{th} Edition (2003) pages 66-667, Wiley-VCH Verlag GmbH & Co. KgaA, Weinheim, Germany). Butenes can also be useful intermediates for the production of isooctanes and isooctenes, which can then be used as intermediates for the production of xylenes, particularly p-xylene. The production of p-xylene is detailed in co-owned U.S. Pat. No. 7,067,708. U.S. Pat. Publ. No. 2005/0228203, and U.S. Pat. Publ. No. 2005/0228204.

### SUMMARY OF THE INVENTION

Disclosed herein is a process for making at least one butene, comprising:
a) contacting a reactant comprising wet ethanol with a base catalyst to make a first reaction product comprising 1-butanol;
b) recovering from the first reaction product a partially-purified first reaction product consisting essentially of 1-butanol and no more than 5 weight percent water based on the weight of the 1-butanol and water combined;
c) contacting the partially-purified first reaction product of step (b), optionally in the presence of a solvent, with at least one acid catalyst at a temperature of about 50 degrees C. to about 450 degrees C. and a pressure from about 0.1 MPa to about 20.7 MPa to produce a second reaction product comprising at least one butene; and
d) recovering said at least one butene from said second reaction product to obtain at least one recovered butene.

In a preferred embodiment, the ethanol of step a) above is obtained from a fermentation broth.

The butenes so produced can be used to produce derivatives such as isoalkanes, C₁₀ to C₁₃ substituted aromatic compounds, butyl alkyl ethers, isooctenes, isooctanes, isooctanols, and isooctyl alkyl ethers. The isooctanes and isooctenes can be further converted to p-xylene. The p-xylene can be further converted to terephthalic acid, a component of polyesters.

### DETAILS

The present invention relates to a process for making butenes from aqueous ethanol via dry butanol. The expression “dry 1-butanol” as used in the present specification and claims denotes a material that is predominantly 1-butanol, but may contain small amounts of water (under about 5% by weight relative to the weight of the 1-butanol plus the water), and may contain small amounts of other materials, such as acetone and ethanol, as long as they do not materially affect the catalytic reaction previously described when performed with reagent grade 1-butanol. The expression “consisting essentially of” means herein that the 1-butanol may include small amounts of other components as long as they do not affect substantially the performance of combined 1-butanol and water in subsequent process steps.

The aqueous ethanol can be obtained from any convenient source, including fermentation using microbiological processes known to those skilled in the art. The fermentative microorganism and the source of the substrate are not critical for the purposes of this invention. The result of the fermentation is a fermentation broth, which is then refined to produce a stream of aqueous ethanol. The refining process may comprise at least one distillation column to produce a first overhead stream that comprises ethanol and water. If the first distillation column is insufficient to produce a first overhead stream with a desired ethanol content, then the first overhead stream can be introduced into a second distillation column to produce a second overhead stream, and so on, ultimately leading to the aqueous ethanol (having at least 5% water) required as the reactant in the present invention. These streams, which are vaporous, can be used directly in the current process, or can be condensed and revaporized for use at a later time.

The stream of aqueous ethanol (which may be diluted with an inert gas such as nitrogen and carbon dioxide) is contacted with at least one base (or basic) catalyst in the vapor or liquid phase at a temperature of about 150 degrees C. to about 500 degrees C. and a pressure from about 0.1 MPa to about 20.7 MPa to produce a first reaction product comprising water and butanol. Typically the first reaction product will also comprise unreacted ethanol, a variety of organic products, and water. The organic products include butanols, predominantly 1-butanol.

The at least one base catalyst can be a homogeneous or heterogeneous catalyst. Homogeneous catalysis is catalysis in which all reactants and the catalyst are molecularly dispersed in one phase. Homogeneous base catalysts include, but are not limited to, alkali metal hydroxides.

Heterogeneous catalysis refers to catalysis in which the catalyst constitutes a separate phase from the reactants and products. See, for example, Hattori, H. (Chem. Rev. (1995) 95:537-550) and Solid Acid and Base Catalysts (Tanabe, K., in Catalysis: Science and Technology, Anderson, J. and Boudart, M (eds.) 1981 Springer-Verlag, New York) for a description of solid catalysts and how to determine whether a particular catalyst is basic.

A suitable base catalyst useful in the current process is either a substance which has the ability to accept protons as defined by Brönsted, or as a substance which has an unshared electron pair with which it can form a covalent bond with an atom, molecule or ion as defined by Lewis.

Examples of suitable base catalysts may include, but may not be limited to, metal oxides, hydroxides, carbonates, silicates, phosphates, aluminates and combinations thereof. Preferred base catalysts may be metal oxides, carbonates, silicates, and phosphates. Preferred metals of the aforementioned compounds may be selected from Group 1, Group 2, and rare earth elements of the Periodic Table. Particularly preferred metals may be cesium, rubidium, calcium, magnesium, lithium, barium, potassium and lanthanum.

The base catalyst may be supported on a catalyst support, as is common in the art of catalysis. Suitable catalyst supports may include, but may not be limited to, alumina, titania, silica, zirconia, zeolites, carbon, clays, double-layered hydroxides, hydrotalcites and combinations thereof. Any method known in the art to prepare the supported catalyst can be used. One method for preparing supported catalysts is to dissolve a metal carboxylate salt in water. A support such as silica is wet with the solution, then calcined. This process converts the supported metal carboxylate to the metal oxide, carbonate, hydroxide or combination thereof. The support can be neutral, acidic or basic, as long as the surface of the catalyst/support combination is basic. Commonly used techniques for treatment of supports with metal catalysts can be found in B. C. Gates, Heterogeneous Catalysis, Vol. 2, pp. 1-29, Ed. B. L. Shapiro, Texas A & M University Press, College Station, Tex., 1984.

The base catalysts of the present invention may further comprise catalyst additives and promoters that will enhance the efficiency of the catalyst. The relative percentage of the catalyst promoter may vary as desired. Promoters may be selected from the Group 8 metals of the Periodic Table, as well as copper and chromium.

The base catalysts of the invention can be obtained commercially, or can be prepared from suitable starting materials using methods known in the art. The catalysts employed for the current invention may be used in the form of powders, granules, or other particulate forms. Selection of an optimal average particle size for the catalyst will depend upon such process parameters as reactor residence time and desired reactor flow rates.

Examples of methods of using base catalysts to convert ethanol to butanol are discussed in the following references.

M. N. Dvornikoff and M. W. Farrar, J. of Organic Chemistry (1957), 11, 540-542, disclose the use of MgO—K₂CO₃—CuCrO₂ catalyst system to promote ethanol condensation to higher alcohols, including 1-butanol. The disclosed liquid phase reaction using this catalyst showed a 13% conversion of ethanol and 47% selectivity to 1-butanol.

U.S. Pat. No. 5,300,695, assigned to Amoco Corp., discloses processes in which an alcohol having X carbon atoms is reacted over an L-type zeolite catalyst to produce a higher molecular weight alcohol. In some embodiments, a first alcohol having X carbon atoms is condensed with a second alcohol having Y carbon atoms to produce an alcohol having X+Y carbons. In one specific embodiment, ethanol is used to produce butanol using a potassium L-type zeolite.

J. I. DiCosimo, et al., in Journal of Catalysis (2000), 190 (2), 261-275, describe the effect of composition and surface properties on alcohol-coupling reactions using Mg_{y}AlOₓ catalysts for alcohol reactions, including ethanol. Also condensation reactions on Mg_{y}AlOₓ samples involved the formation of a carbanion intermediate on Lewis acid-strong Brönsted base pair sites and yielded products containing a new C—C bond, such as n-C₄H₈O (or n-C₄H₉OH) and iso-C₄H₈O (or iso-C₄H₉OH). They also describe, in Journal of Catalysis (1998), 178 (2), 499-510, that the oxidation to acetaldehyde and the aldol condensation to n-butanol both involve initial surface ethoxide formation on a Lewis acid-strong base pair.

PCT Publ. No. WO 2006059729 (assigned to Kabushiki Kaisha Sangi) describes a clean process for efficiently producing, from ethanol as a raw material, higher molecular weight alcohols having an even number of carbon atoms, such as 1-butanol, hexanol and the like. The higher molecular weight alcohols are yielded from ethanol as a starting material with the aid of a calcium phosphate compound, e.g., hydroxyapatite Ca₁₀(PO₄)₆(OH)₂, tricalcium phosphate Ca₃(PO₄)₂, calcium monohydrogen phosphate CaHPO₄×(0-2)H₂O, calcium diphosphate Ca₂P₂O₇, octacalcium phosphate Ca₈H₂(PO₄)₆×5H₂O, tetracalcium phosphate Ca₄(PO₄)₂O, or amorphous calcium phosphate Ca₃(PO₄)₂×nH₂O, preferably hydroxyapatite, as a catalyst, the contact time being 0.4 second or longer.

The catalytic conversion of the wet ethanol to the first reaction product comprising 1-butanol and water can be run in either batch or continuous mode as described, for example, in H. Scott Fogler, (Elements of Chemical Reaction Engineering, 2^{nd} Edition, (1992) Prentice-Hall Inc, CA). Suitable reactors include fixed-bed, adiabatic, fluid-bed, transport bed, and moving bed. During the course of the reaction, the catalyst may become fouled, and therefore it may be necessary to regenerate the catalyst. Preferred methods of catalyst regeneration include, contacting the catalyst with a gas such as, but not limited to, air, steam, hydrogen, nitrogen or combinations thereof, at an elevated temperature.

One skilled in the art will know that conditions, such as temperature, catalytic metal, support, reactor configuration and time can affect the reaction kinetics, product yield and product selectivity. Standard experimentation can be used to optimize the yield of 1-butanol from the reaction.

The first reaction product is then subjected to a suitable refining process to produce a partially-purified first reaction product consisting essentially of 1-butanol and no more than 5 weight percent water, based on the weight of the 1-butanol and water combined. An example of a suitable refining process may include phase separation (depending on the product mix) followed by distillation of the organic phase to recover a material whose organic content is predominantly 1-butanol. If the material contains more than 5 weight percent water, the material can be further distilled to reduce the water content to no more than 5 weight percent, thereby producing the partially-purified first reaction product.

In its first aspect, the present invention relates to a process for making at least one butene comprising contacting the partially-purified first reaction product consisting essentially of 1-butanol and no more than about 5 weight percent water based on the weight of the 1-butanol and water combined with at least one acid catalyst to produce a second reaction product comprising at least one butene, and recovering said at least one butene from said second reaction product to obtain at least one recovered butene. The term “butene” includes 1-butene, isobutene, and/or cis and trans 2-butene.

The reaction to form at least one butene is performed at a temperature of from about 50 degrees Celsius to about 450 degrees Celsius. In a more specific embodiment, the temperature is from about 100 degrees Celsius to about 250 degrees Celsius.

The reaction can be carried out under an inert atmosphere at a pressure of from about atmospheric pressure (about 0.1 MPa) to about 20.7 MPa. In a more specific embodiment, the pressure is from about 0.1 MPa to about 3.45 MPa. Suitable inert gases include nitrogen, argon and helium.

The reaction can be carried out in liquid or vapor phase and can be run in either batch or continuous mode as described, for example, in H. Scott Fogler, (Elements of Chemical Reaction Engineering, 2^{nd} Edition, (1992) Prentice-Hall Inc, CA).

The at least one acid catalyst can be a homogeneous or heterogeneous catalyst. Homogeneous catalysis is catalysis in which all reactants and the catalyst are molecularly dispersed in one phase. Homogeneous acid catalysts include, but are not limited to inorganic acids, organic sulfonic acids, heteropolyacids, fluoroalkyl sulfonic acids, metal sulfonates, metal trifluoroacetates, compounds thereof and combinations thereof. Examples of homogeneous acid catalysts include sulfuric acid, fluorosulfonic acid, phosphoric acid, p-toluenesulfonic acid, benzenesulfonic acid, hydrogen fluoride, phosphotungstic acid, phosphomolybdic acid, and trifluoromethanesulfonic acid.

Heterogeneous catalysis refers to catalysis in which the catalyst constitutes a separate phase from the reactants and products. Heterogeneous acid catalysts include, but are not limited to 1) heterogeneous heteropolyacids (HPAs), 2) natural clay minerals, such as those containing alumina or silica, 3) cation exchange resins, 4) metal oxides, 5) mixed metal oxides, 6) metal salts such as metal sulfides, metal sulfates, metal sulfonates, metal nitrates, metal phosphates, metal phosphonates, metal molybdates, metal tungstates, metal borates, 7) zeolites, and 8) combinations of groups 1-7. See, for example, Solid Acid and Base Catalysts, pages 231-273 (Tanabe, K., in Catalysis: Science and Technology, Anderson, J. and Boudart, M (eds.) 1981 Springer-Verlag, New York) for a description of solid catalysts.

The heterogeneous acid catalyst may also be supported on a catalyst support. A support is a material on which the acid catalyst is dispersed. Catalyst supports are well known in the art and are described, for example, in Satterfield, C. N. (Heterogeneous Catalysis in Industrial Practice, 2^{nd} Edition, Chapter 4 (1991) McGraw-Hill, New York).

In one embodiment of the invention, the reaction is carried out using a heterogeneous catalyst, and the temperature and pressure are chosen so as to maintain the reactant and reaction product in the vapor phase. In a more specific embodiment, the reactant is obtained from a fermentation broth that is subjected to distillation to produce a vapor phase having at least about 42% water. The vapor phase is directly used as a reactant in a vapor phase reaction in which the acid catalyst is a heterogeneous catalyst, and the temperature and pressure are chosen so as to maintain the reactant and reaction product in the vapor phase. It is believed that this vapor phase reaction would be economically desirable because the vapor phase is not first cooled to a liquid prior to performing the reaction.

One skilled in the art will know that conditions, such as temperature, catalytic metal, support, reactor configuration and time can affect the reaction kinetics, product yield and product selectivity. Standard experimentation can be used to optimize the yield of 1-butanol from the reaction.

Following the reaction, if necessary, the catalyst can be separated from the reaction product by any suitable technique known to those skilled in the art, such as decantation, filtration, extraction or membrane separation (see Perry, R. H. and Green, D. W. (eds), Perry's Chemical Engineer's Handbook, 7^{th} Edition, Section 13, 1997, McGraw-Hill, New York, Sections 18 and 22). The at least one recovered butene is useful as an intermediate for the production of linear, low density polyethylene (LLDPE) or high density polyethylene (HDPE), as well as for the production of transportation fuels and fuel additives. For example, butenes can be used to produce alkylate, a mixture of highly branched alkanes, mainly isooctane, having octane numbers between 92 and 96 RON (research octane number) (Kumar, P., et al (Energy & Fuels (2006) 20:481-487). In some refineries, isobutene is converted to methyl t-butyl ether (MTBE). In addition, butenes are useful for the production of alkyl aromatic compounds. Butenes can also be dimerized to isooctenes and further converted to isooctanes, isooctanois and isooctyl alkyl ethers that can be used as fuel additives to enhance the octane number of the fuel.

In its second aspect, the present invention involves contacting the at least one recovered butene with at least one straight-chain, branched or cyclic C₃ to C₅ alkane in the presence of at least one acid catalyst to produce a reaction product comprising at least one isoalkane. Methods for the alkylation of olefins are well known in the art and process descriptions can be found in Kumar, P., et al (supra) for the alkylation of isobutane and raffinate II (a mixture comprising primarily butanes and butenes); and U.S. Pat. No. 6,600,081 (Column 3, lines 42 through 63) for the reaction of isobutane and isobutylene to produce trimethylpentanes (TMPs). Generally, the acid catalysts useful for these reactions have been homogeneous catalysts, such as sulfuric acid or hydrogen fluoride, or heterogeneous catalysts, such as zeolites, heteropolyacids, metal halides, Bronsted and Lewis acids on various supports, and supported or unsupported organic resins. The reaction conditions and product selectivity are dependent on the catalyst. Generally, the reactions are carried out at a temperature between about −20 degrees C. and about 300 degrees C., and at a pressure of about 0.1 MPa to about 10 MPa.

The at least one isoalkane produced by the reaction can be recovered by distillation (see Seader, J. D., supra) and added to a transportation fuel. Unreacted butenes or alkanes can be recycled and used in subsequent reactions to produce isoalkanes.

In its third aspect, the present invention involves contacting the at least one recovered butene with benzene, a C₁ to C₃ alkyl-substituted benzene, or combination thereof, in the presence of at least one acid catalyst or at least one basic catalyst to produce a reaction product comprising at least one C₁₀ to C₁₃ substituted aromatic compound. C₁ to C₃ alkyl-substituted benzenes include toluene, xylenes, ethylbenzene and trimethyl benzene.

Methods for the alkylation of aromatic compounds are well known in the art; discussions of such reactions can be found in Handbook of Heterogeneous Catalysis, Volume 5, Chapter 4 (Ertl, G., Knözinger, H., and Weitkamp, J. (eds), 1997, VCH Verlagsgesellschaft mbH, Weinheim, Germany) and Vora, B. V., et al (Alkylation, in Kirk-Othmer Encyclopedia of Chemical Technology, Volume 2, pages 169-203, John Wiley & Sons, Inc., New York).

In the alkylation of aromatic compounds, acid catalysts promote the addition of butenes to the aromatic ring itself. Typical acid catalysts are homogenous catalysts, such as sulfuric acid, hydrogen fluoride, phosphoric acid, AlCl₃ and boron fluoride, or heterogeneous catalysts, such as alumino-silicates, clays, ion-exchange resins, mixed oxides, and supported acids. Examples of heterogeneous catalysts include ZSM-5, Amberlyst® (Rohm and Haas, Philadelphia, Pa.) and Nafion®-silica (DuPont, Wilmington, Del.).

In base-catalyzed reactions, the butenes are added to the alkyl group of an aromatic compound. Typical basic catalysts are basic oxides, alkali-loaded zeolites, organometallic compounds such as alkyl sodium, and metallic sodium or potassium. Examples include alkali-cation-exchanged X- and Y-type zeolites, magnesium oxide, titanium oxide, and mixtures of either magnesium oxide or calcium oxide with titanium dioxide.

The at least one C₁₀ to C₁₃ substituted aromatic compound produced by the reaction can be recovered by distillation (see Seader, J. D., supra) and added to a transportation fuel. Unreacted butenes, benzene or alkyl-substituted benzene can be recycled and used in subsequent reactions to produce substituted aromatic compounds.

In its fourth aspect, the present invention involves contacting the at least one recovered butene with methanol, ethanol, a C₃ to C₁₅ straight-chain, branched or cyclic alcohol, or a combination thereof, in the presence of at least one acid catalyst, to produce a reaction product comprising at least one butyl alkyl ether. The “butyl” group can be 1-butyl, 2-butyl or isobutyl, and the “alkyl” group can be straight-chain, branched or cyclic. The reaction of alcohols with butenes is well known and is described in detail by Stüwe, A. et al (Handbook of Heterogeneous Catalysis, Volume 4, Section 3.11, pages 1986-1998 (Ertl, G., Knözinger, H., and Weitkamp, J. (eds), 1997, VCH Verlagsgesellschaft mbH, Weinheim, Germany)) for the production of methyl-t-butyl ether (MTBE) and methyl-t-amyl ether (TAME). In general, butenes are reacted with alcohols in the presence of an acid catalyst, such as an ion exchange resin. The etherification reaction can be carried out at pressures of about 0.1 to about 20.7 MPa, and at temperatures from about 50 degrees Celsius to about 200 degrees Celsius.

The at least one butyl alkyl ether produced by the reaction can be recovered by distillation (see Seader, J. D., supra) and added to a transportation fuel. Unreacted butenes or alcohols can be recycled and used in subsequent reactions to produce butyl alkyl ether.

In its fifth aspect, the present invention involves dimerizing the at least one recovered butene to isooctenes, and further converting them to isooctanes, isooctanols or isooctyl alkyl ethers, which are useful fuel additives. The terms isooctenes, isooctanes and isooctanols are all meant to denote eight-carbon compounds having at least one secondary or tertiary carbon. The term isooctyl alkyl ether is meant to denote a compound, the isooctyl moiety of which contains eight carbons, at least one carbon of which is a secondary or tertiary carbon.

The dimerization reaction can be carried out as described in U.S. Pat. No. 6,600,081 (Column 3, lines 42 through 63) for the reaction of isobutane and isobutylene to produce trimethylpentanes (TMPs). The at least one recovered butene is contacted with at least one dimerization catalyst (for example, silica-alumina) at moderate temperatures and pressures and high throughputs to produce a reaction product comprising at least one isooctene. Typical operations for a silica-alumina catalyst involve temperatures of about 150 degrees Celsius to about 200 degrees Celsius, pressures of about 2200 kPa to about 5600 kPa, and liquid hourly space velocities of about 3 to 10. Other known dimerization processes use either hydrogen fluoride or sulfuric acid catalysts. With the use of the latter two catalysts, reaction temperatures are kept low (generally from about 15 degrees Celsius to about 50 degrees Celsius with hydrogen fluoride and from about 5 degrees Celsius to about 15 degrees Celsius with sulfuric acid) to ensure high levels of conversion. Following the reaction, the at least one isooctene can be separated from a solid dimerization catalyst, such as silica-alumina, by any suitable method, including decantation. The at least one isooctene can be recovered from the reaction product by distillation (see Seader, J. D., supra) to produce at least one recovered isooctene. Unreacted butenes can be recycled and used in subsequent reactions to produce isooctenes.

In its sixth aspect, the present invention involves contacting the at least one recovered isooctene produced by the dimerization reaction with at least one hydrogenation catalyst in the presence of hydrogen to produce a reaction product comprising at least one isooctane. Suitable solvents, catalysts, apparatus, and procedures for hydrogenation in general can be found in Augustine, R. L. (Heterogeneous Catalysis for the Synthetic Chemist, Marcel Decker, New York, 1996, Section 3); the hydrogenation can be performed as exemplified in U.S. Patent Application No. 2005/0054861, paragraphs 17-36). In general, the reaction is performed at a temperature of from about 50 degrees Celsius to about 300 degrees Celsius, and at a pressure of from about 0.1 MPa to about 20 MPa. The principal component of the hydrogenation catalyst may be selected from metals from the group consisting of palladium, ruthenium, rhenium, rhodium, iridium, platinum, nickel, cobalt, copper, iron, osmium; compounds thereof; and combinations thereof. The catalyst may be supported or unsupported. The at least one isooctane can be separated from the hydrogenation catalyst by any suitable method, including decantation. The at least one isooctane can then be recovered (for example, if the reaction does not go to completion or if a homogeneous catalyst is used) from the reaction product by distillation (see Seader, J. D., supra) to obtain a recovered isooctane, and added to a transportation fuel. Alternatively, the reaction product itself can be added to a transportation fuel. If present, unreacted isooctenes can be used in subsequent reactions to produce isooctanes.

In its seventh aspect, the present invention involves contacting the at least one recovered isooctene produced by the dimerization reaction with water in the presence of at least one acidic catalyst to produce a reaction product comprising at least one isooctanol. The hydration of olefins is well known, and a method to carry out the hydration using a zeolite catalyst is described in U.S. Pat. No. 5,288,924 (Column 3, line 48 to Column 7, line 66), wherein a temperature of from about 60 degrees Celsius to about 450 degrees Celsius and a pressure of from about 700 kPa to about 24,500 kPa are used. The water to olefin ratio is from about 0.05 to about 30. Where a solid acid catalyst is used, such as a zeolite, the at least one isooctanol can be separated from the at least one acid catalyst by any suitable method, including decantation. The at least one isooctanol can then be recovered from the reaction product by distillation (see Seader, J. D., supra), and added to a transportation fuel. Alternatively, the reaction product itself can be added to a transportation fuel. Unreacted isooctenes, if present, can be used in subsequent reactions to produce isooctanols.

In its eighth aspect, the present invention involves contacting the at least one recovered isooctene produced by the dimerization reaction with at least one acid catalyst in the presence of at least one straight-chain or branched C₁ to C₅ alcohol to produce a reaction product comprising at least one isooctyl alkyl ether. One skilled in the art will recognize that C₁ and C₂ alcohols cannot be branched. The etherification reaction is described by Stüwe, A., et al (Synthesis of MTBE and TAME and related reactions, Section 3.11, in Handbook of Heterogeneous Catalysis, Volume 4, (Ertl, G., Knözinger, H., and Weitkamp, J. (eds), 1997, VCH Verlagsgesellschaft mbH, Weinheim, Germany)) for the production of methyl-t-butyl ether. The etherification reaction is generally carried out at temperature of from about 50 degrees Celsius to about 200 degrees Celsius at a pressure of from about 0.1 to about 20.7 MPa. Suitable acid catalysts include, but are not limited to, acidic ion exchange resins. Where a solid acid catalyst is used, such as an ion-exchange resin, the at least one isooctyl alkyl ether can be separated from the at least one acid catalyst by any suitable method, including decantation. The at least one isooctyl alkyl ether can then be recovered from the reaction product by distillation (see Seader, J. D., supra) to obtain a recovered isooctyl alkyl ether, and added to a transportation fuel. Alternatively, the reaction product itself can be added to a transportation fuel. If present, unreacted isooctenes can be used in subsequent reactions to produce isooctyl alkyl ethers.

In a ninth aspect, the present invention involves contacting recovered isooctene (as obtained from the dimerization of butenes, see the fifth aspect) with hydrogen in the presence of at least one hydrogenation catalyst to produce a reaction product comprising at least one isooctane and recovering the at least one isooctane from the reaction product to obtain at least one recovered isooctane. The recovered isooctane is then contacted with a heterogeneous dehydrocyclization catalyst to produce a reaction product comprising p-xylene, and recovering said p-xylene. Suitable catalysts for the catalytic dehydrocyclization of isooctane to p-xylene are generally described in U.S. Pat. No. 7,067,708 (see column 2, line 51 through column 3, line 21. The temperature can be from about 300 degrees to about 700 degrees Celsius, and pressures can be from about atmospheric pressure to about 1 MPa.

In a tenth aspect, the present invention involves contacting recovered isooctene (see fifth aspect) with a heterogeneous dehydrogenation catalyst to produce a reaction product comprising p-xylene. Suitable dehydrogenation catalysts are generally described in US Pat. Publ. No. 2005/0228204 A1 (see paragraphs [0018-0021]). Suitable temperatures are from about 300 degrees to about 700 degrees Celsius, and pressures can be from about atmospheric pressure to about 1 MPa.

Once recovered, the p-xylene thus formed can be subsequently employed in the production of a variety of other products, including but not limited to terephthalic acid and polyesters.

The use of p-xylene to produce terephthalic acid is well known in the art. See, for example, C-M Park and R. J. Sheehan in “Phthalic Acids and Other Benzenepolycarboxylic Acids”, Kirk-Othmer Encyclopedia of Chemical Technology, John Wiley & Sons, Inc., 2001. In the “Amoco” process described therein, terephthalic acid is produced by catalytic, liquid-phase air oxidation of p-xylene. The catalysts used are generally multivalent heavy metal or metals comprising cobalt. The most popular form of this process uses cobalt and manganese as the multivalent heavy-metal catalysts and bromine as the renewable source for free radicals.

### General Methods and Materials

In the following examples, “C” is degrees Celsius, “mg” is milligram; “ml” is milliliter; “m” is meter, “mm” is millimeter, “min” is minute, “temp” is temperature; “MPa” is mega Pascal; “GC/MS” is gas chromatography/mass spectrometry.

Amberlyst® (manufactured by Rohm and Haas, Philadelphia, Pa.), tungstic acid, 1-butanol and H₂SO₄ were obtained from Alfa Aesar (Ward Hill, Mass.); CBV-3020E (HZSM-5) was obtained from PQ Corporation (Berwyn, Pa.); Sulfated Zirconia was obtained from Engelhard Corporation (Iselin, N.J.); 13% Nafion®/SiO₂ (SAC-13) can be obtained from Engelhard; and H-Mordenite can be obtained from Zeolyst Intl. (Valley Forge, Pa.). Gamma alumina was obtained from Strem Chemical, Inc. (Newburyport, Mass.).

### General Procedure for the Conversion of 1-Butanol to Butenes

Catalyst was added to 1-butanol (1 ml) in a 2 ml vial equipped with a magnetic stir bar. The vial was sealed with a serum cap perforated with a needle to facilitate gas exchange. The vial was placed in a block heater enclosed in a pressure vessel. The vessel was purged with nitrogen and the pressure was set as indicated below. The block was brought to the indicated temperature and maintained at that temperature for the time indicated. After cooling and venting, the contents of the vial were analyzed by GC/MS using a capillary column (either (a) CP-Wax 58 [Varian; Palo Alto, Calif.], 25 m×0.25 mm, 45 C/6 min, 10 C/min up to 200 C, 200 C/10 min, or (b) DB-1701 [J&W (available through Agilent; Palo Alto, Calif.)], 30 m×0.25 mm, 50 C/10 min, 10 C/min up to 250 C, 250 C/2 min).

The examples below were performed according to this procedure under the conditions indicated for each example.

### EXAMPLES 1-14

### Reaction of 1-Butanol (1-BuOH) with an Acid Catalyst to Produce Butenes

The reactions were carried out for 2 hours at 6.9 MPa of N₂.

| Example | | Temp | 1-BuOH | Butenes |
|---|---|---|---|---|
| Number | Catalyst (50 mg) | (C.) | % Conversion | % Selectivity |
| 1 | H₂SO₄ | 200 | 93.6 | 24.1 |
| 2 | Amberlyst ® 15 | 200 | 65.8 | 18.8 |
| 3 | 13% Nafion ®/SiO₂ | 200 | 39.2 | 3.0 |
| 4 | CBV-3020E | 200 | 86.8 | 9.5 |
| 5 | H-Mordenite | 200 | 69.5 | 21.0 |
| 6 | Tungstic Acid | 200 | 9.3 | 38.6 |
| 7 | Sulfated Zirconia | 200 | 0.4 | 100.0 |
| 8 | H₂SO₄ | 120 | 6.9 | 34.9 |
| 9 | Amberlyst ® 15 | 120 | 1.0 | 47.0 |
| 10 | 13% Nafion ®/SiO₂ | 120 | 0.4 | 70.0 |
| 11 | CBV-3020E | 120 | 1.2 | 60.9 |
| 12 | H-Mordenite | 120 | 1.4 | 80.0 |
| 13 | Tungstic Acid | 120 | 1.2 | 73.9 |
| 14 | Sulfated Zirconia | 120 | 0.9 | 93.4 |

What is claimed is:

1. A process for making at least one butene, comprising:
a) contacting a reactant comprising wet ethanol with a base catalyst to make a first reaction product comprising 1-butanol;
b) recovering from the first reaction product a partially-purified first reaction product consisting essentially of 1-butanol and no more than 5 weight percent water based on the weight of the 1-butanol and water combined;
c) contacting the partially-purified first reaction product of step (b), optionally in the presence of a solvent, with at least one acid catalyst at a temperature of about 50 degrees C. to about 450 degrees C. and a pressure from about 0.1 MPa to about 20.7 MPa to produce a second reaction product comprising at least one butene; and
d) recovering said at least one butene from said second reaction product to obtain at least one recovered butene.

2. The process of claim 1, wherein the reactant of step a) is obtained from an ethanol-containing fermentation broth by a process comprising distilling the fermentation broth to obtain a distillate that comprises ethanol and water, and optionally reducing the water in the distillate to achieve an ethanol concentration in the distillate of between about 50 and about 95 weight percent relative to the weight of the remaining water and ethanol combined.

3. A process for making at least one isoalkane, comprising:
a) contacting a reactant comprising wet ethanol with a base catalyst to make a first reaction product comprising 1-butanol and water;
b) recovering from the first reaction product a partially-purified first reaction product consisting essentially of 1-butanol and no more than 5 weight percent water based on the weight of the 1-butanol and water combined;
c) contacting the partially-purified first reaction product of step (b), optionally in the presence of a solvent, with at least one acid catalyst at a temperature of about 50 degrees C. to about 450 degrees C. and a pressure from about 0.1 MPa to about 20.7 MPa to produce a second reaction product comprising at least one butene;
d) recovering said at least one butene from said second reaction product to obtain at least one recovered butene;
e) contacting said at least one recovered butene with a straight-chain, branched or cyclic C₃ to C₅ alkane in the presence of at least one acid catalyst at a temperature between about −20 degrees C. and about 300 degrees C., and at a pressure of about 0.1 MPa to about 10 MPa, to produce a third reaction product comprising at least one isoalkane; and
f) isolating the at least one isoalkane from the third reaction product to produce a least one recovered isoalkane.

4. A process for making at least one C₁₀ to C₁₃ substituted aromatic compound, comprising:
a) contacting a reactant comprising wet ethanol with a base catalyst to make a first reaction product comprising 1-butanol and water;
b) recovering from the first reaction product a partially-purified first reaction product consisting essentially of 1-butanol and no more than 5 weight percent water based on the weight of the 1-butanol and water combined;
c) contacting the partially-purified first reaction product of step (b), optionally in the presence of a solvent, with at least one acid catalyst at a temperature of about 50 degrees C. to about 450 degrees C. and a pressure from about 0.1 MPa to about 20.7 MPa to produce a second reaction product comprising at least one butene;
d) recovering said at least one butene from said second reaction product to obtain at least one recovered butene;
e) contacting the at least one recovered butene with benzene, a C₁ to C₃ alkyl-substituted benzene, or a combination thereof, in the presence of at least one acid catalyst or at least one basic catalyst or a combination thereof, at a temperature of about 100 degrees C. to about 450 degrees C., and at a pressure of about 0.1 MPa to about 10 MPa to produce a third reaction product comprising at least one C₁₀ to C₁₃ substituted aromatic compound; and
f) isolating the at least one C₁₀ to C₁₃ substituted aromatic compound from the third reaction product to produce at least one recovered C₁₀ to C₁₃ substituted aromatic compound.

5. A process for making at least one butyl alkyl ether, comprising:
a) contacting a reactant comprising wet ethanol with a base catalyst to make a first reaction product comprising 1-butanol and water;
b) recovering from the first reaction product a partially-purified first reaction product consisting essentially of 1-butanol and no more than 5 weight percent water based on the weight of the 1-butanol and water combined;
c) contacting the partially-purified first reaction product of step (b), optionally in the presence of a solvent, with at least one acid catalyst at a temperature of about 50 degrees C. to about 450 degrees C. and a pressure from about 0.1 MPa to about 20.7 MPa to produce a second reaction product comprising at least one butene;
d) recovering said at least one butene from said second reaction product to obtain at least one recovered butene;
e) contacting the at least one recovered butene with methanol, ethanol, a C₃ to C₁₅ straight-chain, branched or cyclic alcohol, or a combination thereof, in the presence of at least one acid catalyst at a temperature of about 50 degrees C. to about 200 degrees C., and at a pressure of about 0.1 MPa to about 20.7 MPa to produce a third reaction product comprising at least one butyl alkyl ether; and
f) isolating the at least one butyl alkyl ether from the third reaction product to produce at least one recovered butyl alkyl ether.

6. A process for making at least one isooctene, comprising:
a) contacting a reactant comprising wet ethanol with a base catalyst to make a first reaction product comprising 1-butanol and water;
b) recovering from the first reaction product a partially-purified first reaction product consisting essentially of 1-butanol and no more than 5 weight percent water based on the weight of the 1-butanol and water combined;
c) contacting the partially-purified first reaction product step (b), optionally in the presence of a solvent, with at least one acid catalyst at a temperature of about 50 degrees C. to about 450 degrees C. and a pressure from about 0.1 MPa to about 20.7 MPa to produce a second reaction product comprising at least one butene;
d) recovering said at least one butene from said second reaction product to obtain at least one recovered butene;
e) contacting the at least one recovered butene with at least one acid catalyst at a temperature of about at a temperature of about 50 degrees C. to about 450 degrees C. and a pressure from about 0.1 MPa to about 20.7 MPa to produce a third reaction product comprising at least one isooctene; and
f) isolating the at least one isooctene from the third reaction product to produce at least one recovered isooctene.

7. A process for making at least one isooctane, comprising:
a) contacting a reactant comprising wet ethanol with a base catalyst to make a first reaction product comprising 1-butanol and water;
b) recovering from the first reaction product a partially-purified first reaction product consisting essentially of 1-butanol and no more than 5 weight percent water based on the weight of the 1-butanol and water combined;
c) contacting the partially-purified first reaction product of step (b), optionally in the presence of a solvent, with at least one acid catalyst at a temperature of about 50 degrees C. to about 450 degrees C. and a pressure from about 0.1 MPa to about 20.7 MPa to produce a second reaction product comprising at least one butene;
d) recovering said at least one butene from said second reaction product to obtain at least one recovered butene;
e) comprising contacting the at least one recovered butene with at least one acid catalyst at a temperature of about 50 degrees C. to about 450 degrees C. and a pressure from about 0.1 MPa to about 20.7 MPa to produce a third reaction product comprising at least one isooctene;
f) isolating the at least one isooctene from the third reaction product to produce at least one recovered isooctene;
g) contacting the at least one recovered isooctene with hydrogen in the presence of at least one hydrogenation catalyst at a temperature of about 50 degrees C. to about 300 degrees C. and a pressure from about 0.1 MPa to about 20.7 MPa to produce a fourth reaction product comprising at least one isooctane; and
h) optionally recovering the at least one isooctane from the fourth reaction product to obtain at least one recovered isooctane.

8. A process for making at least one isooctanol, comprising:
a) contacting a reactant comprising wet ethanol with a base catalyst to make a first reaction product comprising 1-butanol and water;
b) recovering from the first reaction product a partially-purified first reaction product consisting essentially of 1-butanol and no more than 5 weight percent water based on the weight of the 1-butanol and water combined;
c) contacting the partially-purified first reaction product step (b), optionally in the presence of a solvent, with at least one acid catalyst at a temperature of about 50 degrees C. to about 450 degrees C. and a pressure from about 0.1 MPa to about 20.7 MPa to produce a second reaction product comprising at least one butene;
d) recovering said at least one butene from said second reaction product to obtain at least one recovered butene;
e) contacting the at least one recovered butene with at least one acid catalyst at a temperature of about 50 degrees C. to about 450 degrees C. and a pressure from about 0.1 MPa to about 20.7 MPa to produce a third reaction product comprising at least one isooctene;
f) isolating the at least one isooctene from the third reaction product to produce at least one recovered isooctene;
g) contacting the at least one recovered isooctene with water and at least one acid catalyst at a temperature of about 50 degrees C. to about 300 degrees C. and a pressure from about 0.1 MPa to about 20.7 MPa to lo produce a fourth reaction product comprising at least one isooctanol; and
h) recovering the at least one isooctanol from the fourth reaction product to obtain at least one recovered isooctanol.

9. A process for making at least one isooctyl alkyl ether, comprising:
a) contacting a reactant comprising wet ethanol with a base catalyst to make a first reaction product comprising 1-butanol and water;
b) recovering from the first reaction product a partially-purified first reaction product consisting essentially of 1-butanol and no more than 5 weight percent water based on the weight of the 1-butanol and water combined;
c) contacting the partially-purified first reaction product step (b), optionally in the presence of a solvent, with at least one acid catalyst at a temperature of about 50 degrees C. to about 450 degrees C. and a pressure from about 0.1 MPa to about 20.7 MPa to produce a second reaction product comprising at least one butene;
d) recovering said at least one butene from said second reaction product to obtain at least one recovered butene;
e) contacting the at least one recovered butene with at least one acid catalyst at a temperature of about 50 degrees C. to about 450 degrees C. and a pressure from about 0.1 MPa to about 20.7 MPa to produce a third reaction product comprising at least one isooctene;
f) isolating the at least one isooctene from the third reaction product to produce at least one recovered isooctene;
g) contacting the at least one recovered isooctene with at least one straight-chain or branched C₁ to C₅ alcohol and at least one acid catalyst at a temperature of about 50 degrees C. to about 200 degrees C. and a pressure of from about 0.1 MPa to about 20.7 MPa to produce a fourth reaction product comprising at least one isooctyl alkyl ether; and
h) recovering the at least one isooctyl alkyl ether from the fourth reaction product to obtain at least one recovered isooctyl alkyl ether.

10. A process for making p-xylene comprising:
a) contacting a reactant comprising wet ethanol with a base catalyst
at a temperature of about 50 degrees C. to about 450 degrees C. and a pressure from about 0.1 MPa to about 20.7 MPa to produce a first reaction product comprising 1-butanol and water;
b) recovering from the first reaction product a partially-purified first reaction product consisting essentially of 1-butanol and no more than 5 weight percent water based on the weight of the 1-butanol and water combined;
c) contacting the partially purified first reaction product of step (b), optionally in the presence of a solvent, with at least one acid catalyst at a temperature of about 50 degrees C. to about 450 degrees C. and a pressure from about 0.1 MPa to about 20.7 MPa to produce a second reaction product comprising at least one butene;
d) recovering said at least one butene from said second reaction product to obtain at least one recovered butene;
e) contacting said at least one recovered butene with at least one acid catalyst to produce a third reaction product comprising at least one isooctene;
f) contacting said third reaction product with hydrogen in the presence of at least one hydrogenation catalyst to produce a fourth reaction product comprising at least one isooctane;
g) recovering the at least one isooctane from the fourth reaction product to obtain at least one recovered isooctane;
h) contacting said at least one recovered isooctane with a heterogenous dehydrocyclization catalyst at a temperature of about 300 degrees C. to about 700 degrees C. and a pressure from about atmospheric to about 1 MPa to produce a fifth reaction product comprising p-xylene; and
(g) recovering the p-xylene obtained from the fifth reaction product to obtain recovered p-xylene.

11. A process for making at least one p-xylene comprising:
a) contacting a reactant comprising wet ethanol with a base catalyst
at a temperature of about 50 degrees C. to about 450 degrees C. and a pressure from about 0.1 MPa to about 20.7 MPa to produce a first reaction product comprising 1-butanol and water;
b) recovering from the first reaction product a partially-purified first reaction product consisting essentially of 1-butanol and no more than 5 weight percent water based on the weight of the 1-butanol and water combined;
c) contacting the partially-purified first reaction product of step (b), optionally in the presence of a solvent, with at least one acid catalyst at a temperature of about 50 degrees C. to about 450 degrees C. and a pressure from about 0.1 MPa to about 20.7 MPa to produce a second reaction product comprising at least one butene;
d) recovering said at least one butene from said second reaction product to obtain at least one recovered butene;
e) contacting said at least one recovered butene with at least one acid catalyst to produce a third reaction product comprising at least one isooctene;
f) contacting said at least one recovered isooctene with a heterogenous dehydrogenation catalyst at a temperature of about 300 degrees C. to about 700 degrees C. and a pressure from about atmospheric to about 1 MPa to produce a fourth reaction product comprising p-xylene; and
g) recovering the p-xylene obtained from the fourth reaction product to obtain recovered p-xylene.
